(19) United States
(12) Patent Application Publication
(10) Pub. No.: US 2005/0287644 A1
(43) Pub. Date: Dec. 29, 2005

(54) Methods for producing and culturing rabbit-mouse hybridomas and monoclonal antibodies secreted by rabbit-mouse hybridomas

(75) Inventors: Yi-Fang Chiu, Taipei County (TW); Lee-Hsuan Chen, Taipei City (TW); Jen-Pin Huang, Taipei County (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Genesis Biotech Inc.

(21) Appl. No.: 11/129,104

(22) Filed: May 13, 2005

Related U.S. Application Data
(60) Provisional application No. 60/571,440, filed on May 14, 2004.

(51) Int. Cl. C12Q 1/68; C12P 21/04; C12N 5/06

(52) U.S. Cl. 435/70.21; 435/334; 530/388.22

(57) ABSTRACT

Methods for producing rabbit-mouse hybridomas are provided. Methods for culturing rabbit-mouse hybridomas are provided. Monoclonal antibodies secreted from rabbit-mouse hybridomas are provided. Epitopes recognized by anti-PRL-3 antibodies are provided.

This application claims the benefit of U.S. Provisional Application No. 60/571,440, filed May 14, 2004, which is incorporated herein by reference.

### FIELD OF THE INVENTION

Methods for producing rabbit-mouse hybridomas are provided. Methods for culturing rabbit-mouse hybridomas are provided. Monoclonal antibodies secreted from rabbit-mouse hybridomas are provided. Epitopes recognized by anti-PRL-3 antibodies are provided.

### BACKGROUND

Antibodies produced in rabbits have certain advantages over those produced in mice. Rabbits generally produce antibodies that are of higher titer and affinity than antibodies produced in mice. See, e.g., Raybould et al. (1988) Science 240:1788-1790; and Spieker-Polet et al. (1995) Proc. Nat'l Acad. Sci. USA 92:9348-9352. Furthermore, rabbits often produce antibodies against immunogens that fail to stimulate a significant antibody response in mice. Id.

Rabbit-mouse hybridomas that secrete rabbit monoclonal antibodies (mAb) may be produced by fusing rabbit immunocytes, such as splenocytes, with mouse myeloma cells. See, e.g., Raybould et al., supra; U.S. Pat. No. 4,977,081 (issued Dec. 11, 1990); EP Publication No. 0 290 014 A2 (published Nov. 9, 1988); Kuo et al. (1985) Mol. Immunol. 22:351-359; Notenboom et al. (1980) J. Immunogenetics 7:359-368; Yarmush et al. (1980) Proc. Nat'l Acad. Sci. USA 77:2899-2903; Yarmush et al. (1981) J. Immunol. 126:2240-2244; and Verbanac et al. (1993) Hybridoma 12:285-295; However, the fusion rate as well as the stability of rabbit-mouse hybridomas may limit further cloning and expansion of the hybridoma clones. See, e.g., Kuo et al. (1985) Mol. Immunol. 22:351-359; Yarmush et al. (1980) Proc. Nat'l Acad. Sci. USA 77:2899-2903.

### SUMMARY OF THE INVENTION

In certain embodiments, a method of culturing a rabbit-mouse hybridoma is provided. In certain embodiments, the method comprises culturing the hybridoma in a medium comprising IL-6 or a functional equivalent thereof. In certain embodiments, the medium further comprises at least about 1% to about 20% V/V fetal calf serum.

In certain embodiments, a method of producing at least one stable rabbit-mouse hybridoma is provided. In certain embodiments, the method comprises combining rabbit immunocytes with mouse myeloma cells; subjecting the rabbit immunocytes and mouse myeloma cells to conditions that promote the formation of at least one hybridoma; and culturing the at least one hybridoma in a medium comprising IL-6 or a functional equivalent thereof, thereby producing at least one stable rabbit-mouse hybridoma. In certain embodiments, the medium is growth medium comprising at least about 1% to about 20% V/V fetal calf serum. In certain embodiments, the medium is selection medium comprising at least about 1% to about 20% V/V fetal calf serum and HAT. In certain embodiments, the method further comprises transferring the at least one stable rabbit-mouse hybridoma to a growth medium lacking IL-6 or a functional equivalent thereof. In certain embodiments, the medium comprises supernatant from a cell culture that secretes IL-6 or a functional equivalent thereof. In certain embodiments, the cell culture that secretes IL-6 or a functional equivalent thereof is AMJ2-C11 (ATCC CRL-2456).

In certain embodiments, a stable rabbit-mouse hybridoma is provided. In certain embodiments, the stable rabbit-mouse hybridoma is produced by a method that comprises combining rabbit immunocytes with mouse myeloma cells; subjecting the rabbit immunocytes and mouse myeloma cells to conditions that promote the formation of at least one hybridoma; and culturing the at least one hybridoma-in a medium comprising IL-6 or a functional equivalent thereof, thereby producing at least one stable rabbit-mouse hybridoma. In certain embodiments, a rabbit monoclonal antibody secreted by the stable rabbit-mouse hybridoma is provided. In certain embodiments, the rabbit monoclonal antibody specifically binds to PRL-3.

In certain embodiments, a stable rabbit-mouse hybridoma is provided, wherein the stable rabbit-mouse hybridoma is clone #1. In certain embodiments, a rabbit monoclonal antibody secreted by clone #1 is provided. In certain embodiments, a rabbit monoclonal antibody that specifically binds to PRL-3 is provided.

In certain embodiments, sequences of epitopes and their variants recognized by anti-PRL-3 antibodies are provided.

### BRIEF DESCRIPTION OF THE DRAWINGS

FIG. 1 shows SDS-PAGE (FIG. 1A) and western blot analysis (FIG. 1B-1E) of a rabbit monoclonal antibody secreted by a rabbit-mouse hybridoma.

FIG. 2 shows SDS-PAGE and western blot analysis of E. coli expressed recombinant hPRL-3 protein (human PRL-3 protein) and polyclonal antibodies (pAb) against the hPRL-3 protein.

FIG. 3 shows peptide array analysis of epitopes of the hPRL-3 protein using both monoclonal and polyclonal antibodies.

### DETAILED DESCRIPTION OF CERTAIN EXEMPLARY EMBODIMENTS

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

It is to be understood that both the foregoing description and the description that follows are exemplary and explanatory only and are not restrictive of the invention, as claimed. In this application, the use of the singular includes the plural unless specifically stated otherwise. In this application, the word “a” or “an” means “at least one” unless specifically stated otherwise. In this application, the use of “or” means “and/or” unless stated otherwise. Furthermore, the use of the term “including,” as well as other forms, such as “includes” and “included,” is not limiting. Finally, terms such as “element” or “component” encompass both elements or components comprising one unit and elements or components that comprise more than one unit unless specifically stated otherwise.

### A. CERTAIN DEFINITIONS

The term “medium” refers to any cell culture medium.

The term “serum-free medium” refers to any “basal” cell culture medium that lacks animal serum. Those skilled in the art are familiar with various embodiments of serum-free medium.

The term “growth medium” refers to a serum-free medium supplemented with serum in an amount that promotes cell growth or viability. In certain embodiments, serum is provided at a concentration from at least about 1% to about 20% V/V serum, including any concentration within that range. Serum may be derived from any animal, including, but not limited to, cow, horse, pig, rabbit, sheep, goat, or human sources. Serum may be derived from an animal at any stage of development, including fetal development. Serum may also be heat-inactivated. The term “growth medium” can also refer to serum-free medium supplemented with serum substitutes, i.e. cytokines and or growth factors.

The term “selection medium” refers to a growth medium that further comprises one or more agents that select for hybridomas. Such agents include, but are not limited to, HAT (hypoxanthine/aminopterin/thymidine) or HT (hypoxanthine/thymidine).

The term “immunogen” refers to any agent or mixture thereof, including, but not limited to, any peptide, protein, or nucleic acid (such as a plasmid), that is capable of eliciting an immune response in any animal.

The term “interleukin-6” or “IL-6” refers to interleukin-6 from any mammalian source. A “cytokine that is functionally equivalent to IL-6” or a “functional equivalent of IL-6” may include a variant of IL-6, such as a substitution mutant, an insertion mutant, a deletion mutant, or an IL-6 fusion protein, that retains an IL-6 function, for example, in the immune response or hematopoiesis. For exemplary IL-6 functions, see, e.g., Taga (1997) J. Immunol. 15:797-819, particularly pages 800-801, and Liguori et al. (2001) Hybridoma 20:189-198, particularly page 189. A “cytokine that is functionally equivalent to IL-6” or a “functional equivalent of IL-6” may also include a cytokine that, like IL-6, transduces a signal by way of the gp130 receptor. Such cytokines include, but are not limited to, IL-11, leukemia inhibitory factor, oncostatin-M, ciliary neurotrophic factor, and cardiotrophin-1. See, e.g., Taga, supra; and Kurth et al. (1999) J. Immunol. 162:1480-1487.

The term “stable” refers to a rabbit-mouse hybridoma that survives at least one, and preferably more, cloning cycles.

### B. CERTAIN EXEMPLARY COMPOSITIONS OF THE INVENTION

In various embodiments, methods, are provided in which cells are grown or cultured in serum-free-medium, growth medium, or selection medium. In certain embodiments, serum-free medium comprises serum-free medium that lacks L-glutamine, such as RPMI-1640 medium (available, e.g., from HyClone, Logan, Utah). In certain such embodiments, the serum-free medium is further supplemented with one or more amino acids, including, but not limited to, L-glutamine. In certain embodiments, serum-free medium is supplemented with any one or more of the following: carbohydrates, including, but not limited to, glucose; buffering agents and salts, including, but not limited to, sodium bicarbonate, MOPS, and HEPES; and antibiotics, including, but not limited to, penicillin and streptomycin; and vitamins. In certain embodiments, serum-free medium comprises RPMI-1640 supplemented with glucose, sodium bicarbonate, L-glutamine, penicillin, streptomycin, and vitamins. The supplementation of serum-free medium is not limited to the above embodiments. The supplementation of serum-free medium is within the skill of those skilled in the art.

In certain embodiments, growth medium comprises a serum-free medium supplemented with at least about 1% to about 20% V/V fetal calf serum, including any concentration of fetal calf serum within this range. In certain embodiments, the fetal calf serum is heat inactivated. In certain embodiments, growth medium comprises RPMI-1640 supplemented with glucose, sodium bicarbonate, HEPES, L-glutamine, penicillin, streptomycin, vitamins, and at least about 1% to about 20% VN fetal calf serum. In certain embodiments, growth medium comprises one or more growth factors that stimulate the proliferation of lymphoid, myeloma, or hybridoma cells. In certain such embodiments, the growth factor is the cytokine IL-6 or a cytokine that is functionally equivalent to IL-6. Exemplary IL-6 amino acid sequences include, but are not limited to, human IL-6 (SEQ ID NO: 92), mouse IL-6 (SEQ ID NO: 93), canine IL-6 (SEQ ID NO: 94), cow IL-6 (SEQ ID NO: 95), rat IL-6 (SEQ ID NO: 96), sheep IL-6 (SEQ ID NO: 97), cat IL-6 (SEQ ID NO: 98), chicken IL-6 (SEQ ID NO: 99), and Mongolian gerbil IL-6 (SEQ ID NO: 100), see Table 4.

In certain embodiments, selection medium is growth medium that comprises the selective agent HAT. In certain embodiments, selection medium comprises one or more growth factors that stimulate the proliferation of lymphoid, myeloma, or hybridoma cells. In certain such embodiments, the growth factor is the cytokine IL-6 or a cytokine that is functionally equivalent to IL-6. In certain embodiments, selection medium comprises RPMI-1640 medium supplemented with glucose, sodium bicarbonate, HEPES, L-glutamine, penicillin, streptomycin, vitamins, IL-6, HAT, and at least about 1% to about 20% V/V fetal calf serum.

### C. CERTAIN EXEMPLARY METHODS OF THE INVENTION

In certain embodiments, a method of producing stable rabbit-mouse hybridomas is provided. In certain embodiments, a method of culturing rabbit-mouse hybridomas is provided. In certain embodiments, one or more rabbits are immunized with an immunogen or a mixture of immunogens. In certain such embodiments, the rabbit is hyperimmunized. Immunization may be, e.g., intrasplenic or subcutaneous. Those skilled in the art are familiar with suitable regimens for immunization, including, for example, the use of an adjuvant.

In certain embodiments, immunocytes are obtained by isolating cells from one or more lymphoid organs of the immunized rabbits, such as the spleen, lymph nodes, or epithelium-associated lymphoid tissue. In certain such embodiments, cells are isolated from the spleens of immunized rabbits. In certain embodiments, the isolated cells are maintained in serum-free medium.

In certain embodiments, myeloma cells are maintained in growth medium. In certain embodiments, myeloma cells are derived from mouse, although myeloma cells derived from other mammals, such as rats, may be used. Any suitable murine myeloma cell line may be used. In certain embodiments, a suitable murine myeloma cell line is obtained from the American Type Culture Collection (ATCC) (Manassas, Va.). In certain embodiments, selection of a suitable murine myeloma cell line is described, e.g., in U.S. Pat. No. 4,977,081, issued Dec. 11, 1990. In certain embodiments, a suitable murine myeloma cell line yields cells that fuse stably with rabbit immunocytes; express a selectable phenotype; and ideally, do not produce or secrete antibody. See id. Such cells include, but are not limited to, e.g., P3/X63-Ag 8 (ATCC TIB9); 45.6TG1.7 (ATCC CRL 1608); P3/NS/1-Ag4-1.(ATCC TIB8); P3×63 Ag8U.1 (ATC CRL 1597); Sp 2/0-Ag14 (ATCC CRL 1581); and FO (ATCC CRL 1646).

In certain embodiments, isolated rabbit immunocytes and murine myeloma cells are mixed together. In certain embodiments, the rabbit immunocytes and murine myeloma cells are mixed together at a ratio that promotes the formation of hybridomas. Those skilled in the art are familiar with suitable ratios. In certain embodiments, the ratio of isolated rabbit immunocytes to murine myeloma cells in the mixture is, e.g., any ratio from about 1:1 to about 25:1. In certain such embodiments, the ratio of isolated rabbit immunocytes to murine myeloma cells in the mixture is from about 1:1 to about 10:1. In certain embodiments, the mixture is exposed to an agent that promotes cell fusion, as would be familiar to those skilled in the art. Such agents include, but are not limited to, a polyethylene glycol (PEG), such as PEG 4000. After the exposure, the cells are separated from the fusion agent, e.g., by centrifuging the cells, or by any other conventional separation technique.

In certain embodiments, the cells, which have been separated from the fusion agent, are resuspended in selection medium. In certain embodiments, the cells are resuspended in growth medium, followed by gradual replacement of the growth medium with selection medium over the course of 24 to 72 hours after the fusion procedure. In certain embodiments, after being cultured in selection medium for an amount of time suitable to select for hybridomas, the cells are transferred to and cultured in growth medium. In certain such embodiments, selection medium is gradually replaced with growth medium.

In certain embodiments, growth medium or selection medium or both are supplemented with IL-6 or a functional equivalent thereof. In certain embodiments, IL-6 or a functional equivalent thereof is obtained from any mammalian source, native or recombinant. In certain embodiments, IL-6 or a functional equivalent thereof is synthesized, for example, using chemical methods known in the art.

In certain embodiments, growth medium or selection medium or both are supplemented with IL-6 or a functional equivalent thereof at a concentration that improves the stability or stimulates the proliferation of a hybridoma. In certain embodiments, IL-6 or a functional equivalent thereof is provided to a final concentration of about 0.05 ng/ml to about 1 mg/ml. In certain embodiments, IL-6 or a functional equivalent thereof is provided by supplementing the growth medium or selection medium with the supernatant of a cultured cell line that secretes IL-6 or a functional equivalent thereof. In certain such embodiments, the supernatant is provided at any concentration within a range of about 2% to about 15% V/V. In certain such embodiments, the concentration is from about 5% to about 10% V/V. In certain such embodiments, the concentration is about 8% V/V. In certain embodiments, a cultured cell line that secretes IL-6 or a functional equivalent thereof is a murine cell line. In certain such embodiments, the murine cell line is AMJ2-C11 (ATCC CRL-2456) (American Type Culture Collection (ATCC), Manassas, Va.).

In certain embodiments, cells that have been cultured in growth medium or selection medium supplemented with IL-6 or a functional equivalent thereof are transferred to growth medium or selection medium that is not supplemented with IL-6 or a functional equivalent thereof. In certain such embodiments, cells are transferred at any point after the formation of one or more stable hybridomas. In certain embodiments, cells are cultured in growth medium or selection medium supplemented with IL-6 or a functional equivalent thereof for about one to two months before they are transferred to growth medium or selection medium that is not supplemented with IL-6 or a functional equivalent thereof.

In certain embodiments, cells that have been subjected to selection in selection media, and optionally, grown in growth medium, are screened to identify hybridomas secreting a monoclonal antibody to the immunogen. In certain embodiments, the cells are screened by ELISA. In certain embodiments, to facilitate screening, the cells are plated in the wells of one or more multi-well plates. In certain such embodiments, the cells are plated at a concentration of about 10⁴ to about 10¹⁰ cells/ml.

In certain embodiments, a method of culturing a rabbit-mouse hybridoma is provided. In certain embodiments, a rabbit-mouse hybridoma is cultured in a medium supplemented with IL-6 or a functional equivalent thereof. In certain embodiments, a rabbit-mouse hybridoma is cultured in a medium supplemented with at least about 1% to about 20% V/V fetal calf serum.

In certain embodiments, rabbit monoclonal antibodies secreted from a rabbit-mouse hybridoma are provided.

In certain embodiments, sequences of epitopes recognized by anti-PRL-3 antibodies are provided. Also provided are variants of these sequences resulting from one or more amino acid substitutions, deletions, and/or insertions. Such variants are functionally equivalent to the epitopes in that they are still recognized by antibodies to the epitopes, meaning that in an ELISA, antibodies to the epitopes bind to the variants with an O.D.₄₀₅ value that is at least two fold the O.D.₄₀₅ value obtained with a “blank” control, i.e., an ELISA in which no antigen is provided and only first and secondary antibodies are used.

The identified epitopes of PRL-3 can be used for diagnostic purposes for detecting the presence of the PRL-3 protein, which is involved in the metastasis of colorectal cancer. The epitopes can also be used to elicit antibodies that bind to the PRL-3 protein.

The following examples are intended for illustration purposes only and should not be construed as limiting in any way.

### EXAMPLES

Immunization and Isolation of Rabbit Splenocytes

A rabbit was hyperimmunized intrasplenically with the synthetic peptide “GP23.” GP23 is a peptide fragment of the human PRL-3 protein. Human PRL-3 is a protein tyrosine phosphatase involved in the metastasis of colorectal cancer. See Saha et al. (2001) Science 294:1343-1346. GP23 is located in the putative extracellular domain of the human PRL-3 protein. This domain is conserved among a number of vertebrate protein tyrosine phosphatases.

The hyperimmunized rabbit was sacrificed, and the spleen surgically removed. The spleen was immediately placed in warm serum-free medium and homogenized to create a splenocyte suspension. The splenocyte suspension was transferred to centrifuge tubes and centrifuged. The pellets were washed three times with serum free medium. Viable cells were counted and the desired number of cells were selected.

Conditioning of Murine Myelomas

FO (ATCC CRL 1646) myeloma cells were maintained in growth medium. Myeloma cells were harvested and washed three times with serum-free medium. Viable cells were counted and the desired number of cells were selected.

Fusion of Cells and Selection of Hybridomas

The splenocytes and myeloma cells were mixed in a ratio of 1:1 to 10:1. The cells in the mixture were pelleted by centrifugation. The pellet was exposed to PEG 4000 for two minutes, and then serum-free medium was added within five minutes. The mixture was centrifuged to repellet the cells.

The supernatant was removed and the cell pellet resuspended in HAT selection medium supplemented with the culture supernatant of AMJ2-C11 cells. The culture supernatant was provided at a concentration of about 8% V/V. The resuspended cells were transferred into the wells of a 96-well plate in 100 μl aliquots. The plate was then transferred to a 37° C. incubator.

The supernatant from each well was screened for antibody against GP23 in an ELISA assay using diluted horseradish peroxidase conjugated goat anti-rabbit IgG (H+L) and the TMB substrate system. Positive clones were selected and transferred to HAT selection medium supplemented with the culture supernatant of AMJ2-C11 cells, as described above. The positive clones were further cultured. Monoclonal antibody-secreting hybridomas were cloned by limiting dilution.

Analysis of Rabbit Monoclonal Antibody from Hybridoma “Clone #1”

A rabbit-mouse hybridoma, “clone #1,” was isolated by the above procedure. Clone #1 secreted intact rabbit monoclonal antibodies that specifically recognized the GP23 peptide by ELISA (data not shown).

SDS-polyacrylamide gel electrophoresis (SDS-PAGE) and western blots were used to demonstrate that the rabbit monoclonal antibodies secreted by clone #1 recognized recombinant human PRL-3. These experiments also confirmed that clone #1 secreted rabbit monoclonal antibodies, and not mouse monoclonal antibodies.

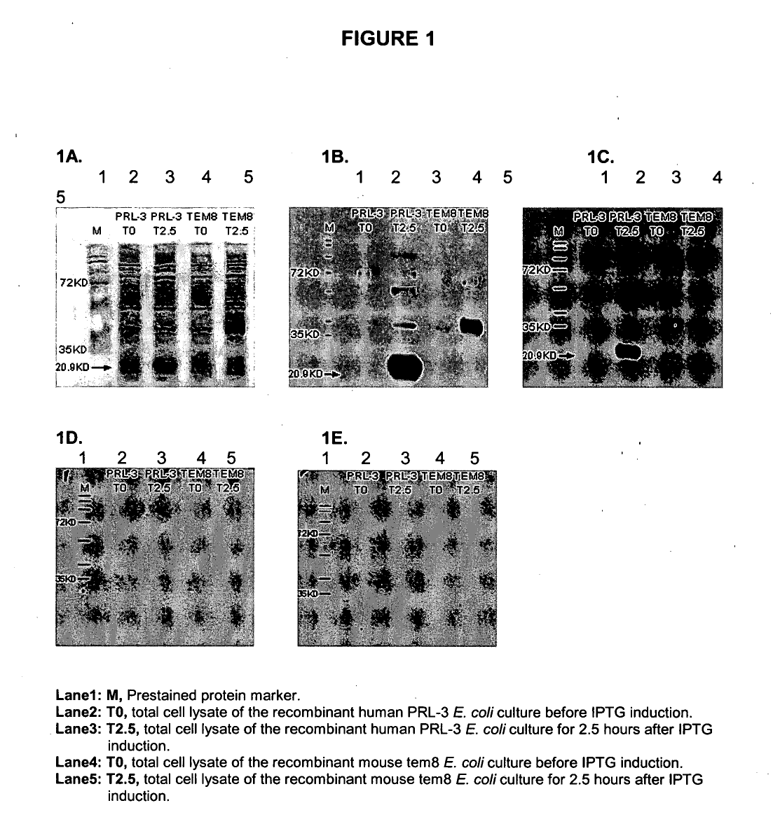

FIG. 1 shows SDS-PAGE and western blot analysis of total cell lysates from E. coli in which expression of recombinant human PRL-3 (lane 3) and recombinant mouse tem8/mixed immunogens (lane 5) was induced. Lanes 2 and 4 show total cell lysates from E. coli prior to induction. Total protein was stained after SDS-PAGE, shown in FIG. 1A.

For the western blot analysis shown in FIGS. 1B and 1D, the primary antibodies used were rabbit polyclonal antibody against human PRL-3 and rabbit polyclonal antibody against tem8. In FIGS. 1C and 1E, the primary antibody used was the monoclonal antibody secreted by clone #1. In FIGS. 1B and 1C, the secondary antibody used was goat anti-rabbit IgG (H+L) conjugated to horseradish peroxidase (HRP). In FIGS. 1D and 1E, the secondary antibody used was goat anti-mouse IgG (H+L) conjugated to horseradish peroxidase. Secondary antibodies were detected using the Amersham ECL Chemiluminescence kit.

FIG. 1B shows that a rabbit polyclonal antibody against human PRL-3 specifically recognized human PRL-3 (lane 3) and was detected using goat anti-rabbit secondary antibody. FIG. 1C shows that the monoclonal antibody secreted by clone #1 also recognized human PRL-3 (lane 3) and,was detected using goat anti-rabbit secondary antibody. FIG. 1E shows that the monoclonal antibody secreted by clone #1 was not recognized by goat anti-mouse secondary antibody. Thus, this experiment shows that the monoclonal antibody secreted by clone #1 is a rabbit monoclonal antibody that specifically recognizes human recombinant PRL-3.

Generation of Polyclonal Antibodies Against Recombinant Human PRL-3

Polyclonal antibodies were raised against recombinant hPRL-3 protein. Western blot analysis was used to confirm that the anti-hPRL-3 antibodies specifically bound to human PRL-3.

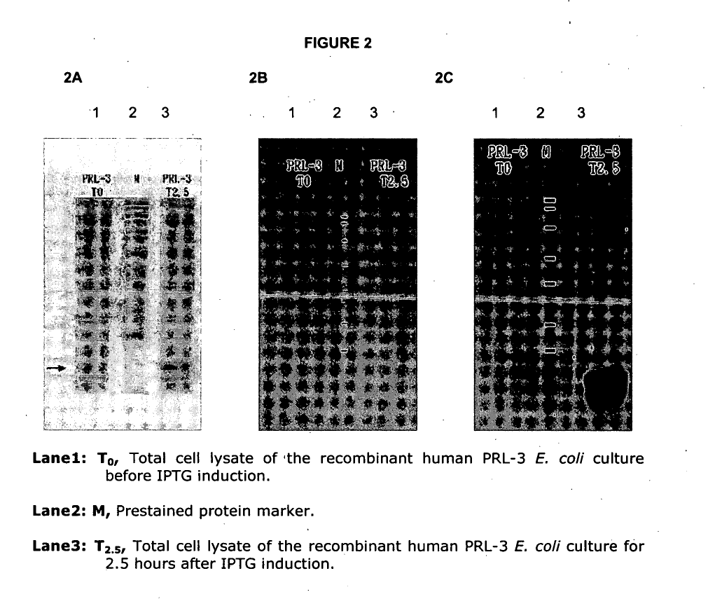

FIG. 2 shows SDS-PAGE and western blot analysis of total cell lysates from E. coli in which expression of recombinant human PRL-3 was induced. FIG. 2A shows the result of SDS-polyacrylamide gel electrophoresis (SDS-PAGE) of E. coli total cell lysate before (Lane 1) and after (Lane 3) induction of hPRL-3 protein. The gel was stained with Commassie brilliant blue. FIGS. 2B and 2C show the results of western blot analysis with rabbit pre-immune serum and rabbit anti-hPRL-3 polyclonal antibodies (generated using the recombinant hPRL-3), respectively. All primary antibodies were diluted 5000× in 3% BSA/PBS. The secondary antibodies used were goat anti-rabbit antibodies conjugated to HRP. Polyclonal antibodies raised against the recombinant hPRL-3 protein recognized human PRL-3 protein as shown in FIG. 2C.

Epitope Mapping of Anti-PRL-3 Antibodies

ELISA Analysis of Polyclonal Antibodies

Polyclonal antibodies were used in the ELISA method to map the epitopes of anti-hPRL-3 antibodies.

Table 1 shows the serial number assigned to different peptides in the human PRL-3 peptide array on the ELISA microtiter plate. Table 2 shows the sequences of the peptides on the array. Columns 1 and 2 of Table 3 show the results of ELISA analysis using polyclonal antibodies raised against synthetic peptides of hPRL-3. Columns 3 and 4 of Table 3 show the results of ELISA analysis using polyclonal antibodies raised against recombinant hPRL-3 protein.

The microtiter plate for ELISA was-coated with synthetic peptides from hPRL-3, as shown in Table 2. The primary antibodies used were polyclonal antibodies obtained from rabbit immunized with GP23 synthetic peptide (The GP23 synthetic peptide corresponds to amino acids 42-69 of hPRL-3, ATTWRVCEVTYDKTPLEKDGITWDWP (SEQ ID NO: 85), which sequence is 100% identical with PRL-3 from other species, such as mouse and rat.) or from rabbit immunized with recombinant hPRL-3 protein.

The results in Table 3 show that both anti-PRL-3 polyclonal antibodies raised from a synthetic PRL-3 peptide and recombinant hPRL-3 protein share the same epitope of PLEKDGITVVDW (SEQ ID NO: 1). See 1 G of column 1 and 3G of column 3 in Tables 2 and 3. The sequence PLEKDGITWDW is located at 57-68 of human PRL-3 (AAP 35967, SEQ ID NO: 87) and mouse PRL-3 (NP 033001, SEQ ID NO: 88). Antibodies raised against recombinant human PRL-3 also detected the epitope INSKQLTYLEKY (SEQ ID NO: 2). See 4D of column 4 in Tables 2 and 3. The sequence INSKQLTYLEKY corresponds to amino acids 116-127 of human PRL-3 (AAP35967, SEQ ID NO: 87), amino acids 141-152 of mouse PRL-3 (NP 033001, SEQ ID NO: 88), and amino acids 811-822 of canine PRL-3 (XP 539183, SEQ ID NO: 89). Antibodies raised against recombinant human PRL-3 also detected the epitope of THNPTNATLSTF (SEQ ID NO: 86). See 3D of column 3 in Tables 2 and 3. The sequence THNPTNATLSTF corresponds to amino acids 22-33 of human PRL-3 (AAP35967, SEQ ID NO: 87) and Gallus gallus PRL-3 (XP 418417.1, SEQ ID NO: 90)

TABLE 1
The design of human PRL-3 peptide array on microtiter plate

| | |
|---|---|
| pAb raised from synthetic | pAb raised from |
| peptide of hPRL-3 | recombinant hPRL-3 |

| | 1 | 2 | 3 | 4 |
|---|---|---|---|---|
| A | GP130-1 | GP130-16 | GP130-1 | GP130-16 |
| B | GP130-2 | GP130-19 | GP130-2 | GP130-19 |
| C | GP130-3 | GP130-20 | GP130-3 | GP130-20 |
| D | GP130-4 | GP130-21 | GP130-4 | GP130-21 |
| E | GP130-5 | GP130-23 | GP130-5 | GP130-23 |
| F | GP130-6 | GP130-24 | GP130-6 | GP130-24 |
| G | GP130-9 | Blank-1Ab | GP130-9 | Blank-1Ab |
| H | GP130-12 | Blank-PBS | GP130-12 | Blank-PBS |

TABLE 2

The peptide sequences ot human PRL-3
peptide array on microtiter plate

| pAb raised from synthetic | | pAb raised from | |
|---|---|---|---|
| peptide of hPRL-3 | | recombinant hPRL-3 | |
| 1 | 2 | 3 | 4 |

| | | | | |
|---|---|---|---|---|
| A | MARMNRPAPVEV | AGLGRAPVLVAL | MARMNRPAPVEV | AGLGRAPVLVAL |
| | (SEQ ID NO:14) | (SEQ ID NO:21) | (SEQ ID NO:26) | (SEQ ID NO:33) |
| B | APVEVSYKHMRF | EDAIQFIRQKRR | APVEVSYKHMRF | EDAIQFIRQKRR |
| | (SEQ ID NO:15) | (SEQ ID NO:22) | (SEQ ID NO:27) | (SEQ ID NO:34) |
| C | KHMRFLITHNPT | RQKRRGAINSKQ | KHMRFLITHNPT | RQKRRGAINSKQ |
| | (SEQ ID NO:16) | (SEQ ID NO:23) | (SEQ ID NO:28) | (SEQ ID NO:35) |
| D | THNPTNATLSTF | INSKQLTYLEKY | THNPTNATLSTF | INSKQLTYLEKY |
| | (SEQ ID NO:17) | (SEQ ID NO:2) | (SEQ ID NO:29) | (SEQ ID NO:2) |
| E | TLSTFIEDLKKY | KQRLRFKDPHTH | TLSTFIEDLKKY | KQRLRFKDPHTH |
| | (SEQ ID NO:18) | (SEQ ID NO:24) | (SEQ ID NO:30) | (SEQ ID NO:36) |
| F | DLKKYGATTVVR | DPHTHKTRCCVM | DLKKYGATTVVR | DPHTHKTRCCVM |
| | (SEQ ID NO:19) | (SEQ ID NO:25) | (SEQ ID NO:31) | (SEQ ID NO:37) |
| G | PLEKDGITVVDW | Blank-1Ab | PLEKDGITVVDW | Blank-^{1st} Ab |
| | (SEQ ID NO:1) | | (SEQ ID NO:1) | |
| H | GKVVEDWLSLVK | Blank-PBS | GKVVEDWLSLVK | Blank-PBS |
| | (SEQ ID NO:20) | | (SEQ ID NO:32) | |

TABLE 3
ELISA data from human PRL-3 peptide array probed
with two PRL-3 polyclonal antibodies

| | O.D.₄₀₅ - pAb raised from | | O.D.₄₀₅ - pAb raised from | |
|---|---|---|---|---|
| | synthetic peptide of hPRL-3 | | recombinant hPRL-3 | |
| | 1 | 2 | 3 | 4 |
| A | 0.190 | 0.227 | 0.169 | 0.191 |
| B | 0.172 | 0.182 | 0.140 | 0.168 |
| C | 0.181 | 0.191 | 0.150 | 0.170 |
| D | 0.178 | 0.155 | 0.241 | 0.626 |
| E | 0.160 | 0.187 | 0.131 | 0.160 |
| F | 0.184 | 0.178 | 0.153 | 0.140 |
| G | 0.994 | 0.128 | 1.471 | 0.098 |
| H | 0.192 | 0.058 | 0.175 | 0.058 |

Analysis of Monoclonal and Polyclonal Antibodies Using Membrane-Based Peitide Arrays

Epitope mapping of monoclonal antibodies from Clone #1 and polyclonal antibodies raised against human PRL-3 recombinant protein was preformed using membrane-based peptide arrays.

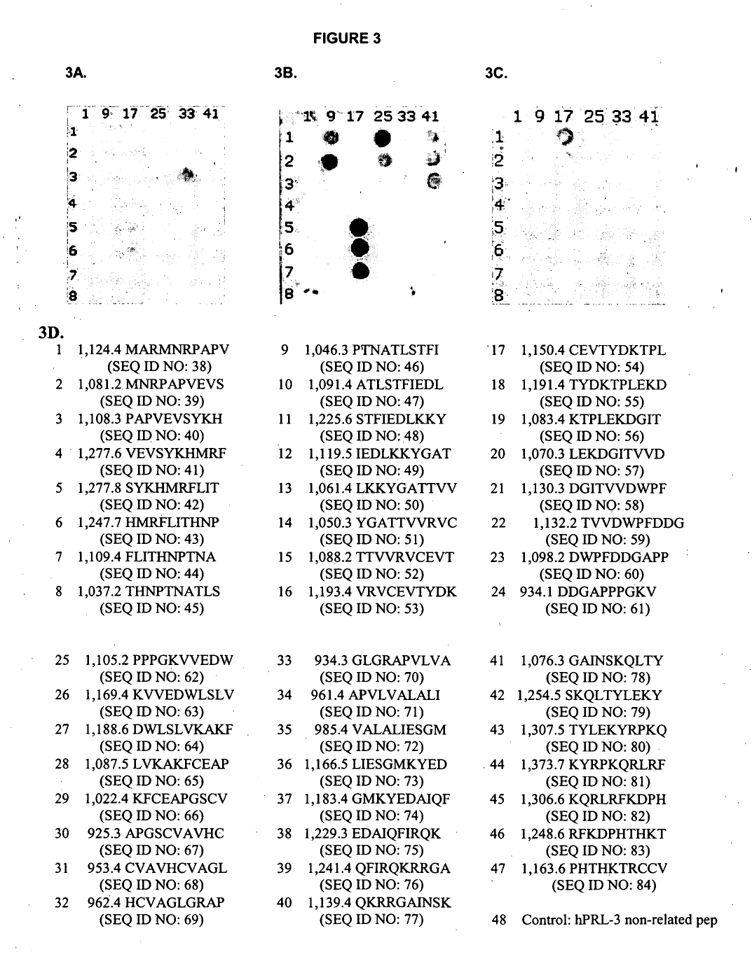

Each peptide array comprises in situ synthesized, overlapping peptides (12-mers) spanning hPRL-3, as shown in FIG. 3D. A control peptide, “hPRL-3 non-related peptide” was also included on the arrays. There are a total of 48 peptide dots on each array, and adjacent peptide dots have partial overlap in amino acid sequences.

A control peptide array was first incubated with rabbit pre-immune serum diluted 5000× in 3% BSA/PBS. The array was then incubated with goat anti-rabbit secondary antibodies conjugated to HRP. FIG. 3A does not show any binding.

A second peptide array was incubated with rabbit polyclonal antibodies raised against hPRL-3 recombinant protein. The rabbit polyclonal antibodies were diluted 5000× in 3% BSA/PBS. The secondary antibody was goat anti-rabbit conjugated to HRP. FIG. 3B shows that the antibodies recognized the following epitopes:
PTNATLSTFIEDL (SEQ ID NO: 3), see FIG. 3D-#9,#10 (amino acids 25-37 of human (AAP35967, SEQ ID NO: 87) PRL-3 and Pan troglodytes (chimpanzee) PRL-3 (XM 519985.1, SEQ ID NO: 91), and amino acids 634-646 of canine PRL-3 (XP 539183, SEQ ID NO: 89)); A preferred sequence is the underlined sequence of NATLSTFI (SEQ ID NO: 4).
DGITWDWPFDDGAPP (SEQ ID NO: 5), see FIG. 3D-#21-#23 (amino acids 61-76 of human PRL-3 (AAP35967, SEQ ID NO: 87) and mouse PRL-3 (NP 033001, SEQ ID NO: 88) and amino acids 634-646 of canine PRL-3 (XP 539183, SEQ ID NO: 89)); A preferred sequence is the underlined sequence of DWPFDDG (SEQ ID NO: 6).
PPPGKVVEDWLSLV (SEQ ID NO: 7), see FIG. 3D-#25-#26 (amino acids 75-88 of human PRL-3 (AAP35967, SEQ ID NO: 87), amino acids 75-87 of mouse PRL-3(NP 033001, SEQ ID NO: 88) and amino acids 745-757 of canine PRL-3 (XP 539183, SEQ ID NO: 89)); A preferred sequence is the underlined sequence of KWEDW (SEQ ID NO: 8).

GAINSKQLTYLEKYRPKQ (SEQ ID NO: 9), see FIG. 3D-#41-#43 (amino acids 114-131 of human PRL-3 (AAP35967, SEQ ID NO: 87), amino acids 139-156 of mouse PRL-3(NP 033001, SEQ ID NO: 88), and amino acids 809-826 of canine PRL-3 (XP 539183, SEQ ID NO: 89)); A preferred sequence is the underlined sequence of TYLEKY (SEQ ID NO: 10).

A third peptide array was incubated with the culture supernatant of clone #1, which secreted anti-GP23 rabbit monoclonal antibody. The culture supernatant was diluted 10× in 3% BSA/PBS. The secondary antibody was goat anti-rabbit conjugated to HRP. FIG. 3C shows that the antibodies recognized the following epitope:
CEVTYDKTPLEKD (SEQ ID NO: 11), see FIG. 3D-#17, #18 (amino acids 49-61 of human PRL-3 (AAP35967, SEQ ID NO: 87) and mouse PRL-3 (NP 033001, SEQ ID NO: 88) and amino acids 658-670 of canine PRL-3 (XP 539183, SEQ ID NO: 89)); A preferred sequence is the underlined sequence of TYDKTPL (SEQ ID NO: 12).

The results from ELISA are consistent with that from membrane-based peptide array mapping. The C-terminal sequence of rabbit anti-PRL-3 mAb epitope (dot #17, #18) shares the same 5 amino acid (PLEKD (SEQ ID NO: 13) (amino acids 57-61 of human (AAP35967, SEQ ID NO: 87) and mouse PRL-3 (NP 033001, SEQ ID NO: 88), and aa 666-670 of canine PRL-3 (XP 539183, SEQ ID NO: 89)) with the N-terminal sequence of anti-hPRL-3 pAb epitope, shown in 1G of column 1 and 3 G of column 3 in Table 2.

Other embodiments consistent with the invention will be apparent to those skilled in the art from consideration of the specification and practice of the invention disclosed herein. It is intended that the specification and examples be considered as exemplary only, with a true scope and spirit of the invention being indicated by the following claims.

TABLE 4
IL-6 Protein Sequences of Different Animals

| | |
|---|---|
| SEQ ID NO:92 | MNSFSTSAFGPVAFSLGLLLVLPAAFPAPVPPGEDSKDVAAPHR |
| Human | QPLTSSERIDKQIRYILDGISALRKETCNKSNMCESSKEALAENNLNLPKMAEKDGCF |
| | QSGFNEETCLVKIITGLLEFEVYLEYLQNRFESSEEQARAVQMSTKVLIQFLQKKAKN |
| | LDAITTPDPTTNASLLTKLQAQNQWLQDMTTHLILRSFKEFLQSSLRALRQM |
| SEQ ID NO:93 | MKFLSARDFHPVAFLGLMLVTTTAFPTSQVRRGDFTEDTTPNRP |
| Mouse | VYTTSQVGGLITHVLWEIVEMRKELCNGNSDCMNNDDALAENNLKLPEIQRNDGCYQT |
| | GYNQEICLLKISSGLLEYHSYLEYMKNNLKDNKKDKARVLQRDTETLIHIFNQEVKDL |
| | HKIVLPTPISNALLTDKLESQKEWLRTKTIQFILKSLEEFLKVTLRSTRQT |
| SEQ ID NO:94 | MNSLSTSAFSLGLLLVMATAFPTPGPLAGDSKDDATSNSLPLTS |
| Canine | ANKVEELIKYILGKISALRKEMCDKFNKCEDSKEALAENNLHLPKLEGKDGCFQSGFN |
| | QETCLTRITTGLVEFQLHLNILQNNYEGDKENVKSVHMSTKILVQMLKSKVKNQDEVT |
| | TPDPTTDASLQAILQSQDECVKHTTIHLILRSLEDFLQFSLRAVRIM |
| SEQ ID NO:95 | MNSRFTSAFTPFAVSLGLLLVMTSAFPTPGPLGEDFKNDTTPGR |
| Cow | LLLTTPEKTEALIKRMVDKISAMRKEICEKNDECESSKETLAENKLNLPKMEEKDGCF |
| | QSGFNQAICLIRTTAGLLEYQIYLDYLQNEYEGNQENVRDLRKNIRTLIQILKQKIAD |
| | LITTPATNTDLLEKMQSSNEWVKNAKIILILRNLENFLQFSLRAIRMK |
| SEQ ID NO:96 | MKFLSARDFQPVAFLGLMLLTATAFPTSQVRRGDFTEDTTHNRP |
| Rat | VYTTSQVGGLITYVLREILEMRKELCNGNSDCMNSDDALSENNLKLPEIQRNDGCFQT |
| | GYNQEICLLKICSGLLEFRFYLEFVKNNLQDNKKDKARVIQSNTETLVHIFKQEIKDS |
| | YKIVLPTPTSNALLMEKLESQKEWLRTKTIQLILKALEEFLKVTMRSTRQT |
| SEQ ID NO:97 | MNSLFTSAFSPLAVSLGLLLVMTSAFPTPGPLGEDFKNDTTPSR |
| Sheep | LLLTTPEKTEALIKHIVDKISAIRKEICEKNDECENSKETLAENKLKLPKMEEKDGCF |
| | QSGFNQAICLIKTTAGLLEYQIYLDFLQNEFEGNQETVMELQSSIRTLIQILKEKIAG |
| | LITTPATHTDMLEKMQSSNEWVKNAKVIIILRSLENFLQFSLRAIRMK |
| SEQ ID NO:98 | MNFLSTSAFSPLAFSLGLLLWATAFPTPGPLGGDATSNRLPLT |
| Cat | PADKMEELIKYILGKISALKKEMCDNYNKCEDSKEALAENNLNLPKLAEKDGCFQSGF |
| | NQETCLTRITTGLQEFQIYLKFLQDKYEGDKENAKSVYTSTNVLLQMLKRKGKNQDEV |
| | TIPVPTVEVGLQLSCSHRRVAEAHNNHLTLRRLEDFLQLRLRAVRIM |
| SEQ ID NO:99 | MNFTEGCEATGRRPGSAGSRRRRAPRPGPVALLPLLLPLLLPPA |
| Chicken | AAVPLPAAADSSGEVGLEEEAGARRALLDCEPLARVLRDRAVQLQDEMCKKFTVCENS |
| | MEMLVRNNLNLPKVTEEDGCLLAGFDEEKCLTKLSSGLFAFQTYLEFIQETFDSEKQN |
| | VESLCYSTKHLAATIRQMVINPDEWIPDSAAQKSLLANLKSDKDWIEKITMHLILRD |
| | FTSFMEKTVRAVRYLKKTRSFSA |
| SEQ ID NO:100 | ENNLKLPEIQRDDGCFHTGYNRDVCLLKITSGLLEYQTYLEYVK |
| Mongolian | NNLQDNKKDKARAIQSNTKTLIRIFKQEVKDPGQIVFPDPTSEALLLEKLESQTEWLK |
| Gerbil | |

TABLE 5
PRL-3 Protein Sequences of Different Animals

| | | |
|---|---|---|
| | Gene Bank | |
| SEQ ID NO. | Accession Number | Sequence |

| | | | |
|---|---|---|---|
| SEQ ID NO: 87 | AAP35967 | 1 | marmnrpapv evsykhmrfl ithnptnatl stfiedlkky gattvvrvce vtydktplek |
| | (Human) | 61 | dgitvvdwpf ddgapppgkv vedwlslvka kfceapgscv avhcvaglgr krrgainskq |
| | | 121 | ltylekyrpk qrlrtkdpht hktrccvm |
| SEQ ID NO: 88 | NP_033001 | 1 | marmnrpapv evsyrhmrfl ithnpsnatl stfiedlkky gattvvrvce vtydktplek |
| | (Mouse) | 61 | dgitvvdwpf ddgapppgkv vedwislika kfyndpgscv avhcvaglgr apvlvalali |
| | | 121 | esgmkyedai qfirqkrrga inskqltyle kyrpkqrlrf kdphthktrc cvm |
| SEQ ID NO: 89 | XP_539183 | 1 | mplfcvavav egeepfsqga csgpsqagav papgltrgag gqppqasgsp rlherepgar |
| | (Canine) | 61 | alespsprsr tpalpqprrp aaqrcsapgl pgpglrrrpc astgpgagpw plgplapaap |
| | | 121 | alparlgted arapsgsrgt hrtfacsaag grggaaepgd llgaalratr sapdppswgl |
| | | 181 | tsgprrgsfw akrrgahacr gvlvgpapar gpygtccdpc eryhlvgtwm lissipsiss |
| | | 241 | ipsnssipgn psipsipgnp sipsipgnts ipsipsipgn psnssipgnp sipsipgnpa |
| | | 301 | spaspatpvs paewdtwrhq gkpsggneaq slaplvvpgg fsqcrqvqqv srppaadpal |
| | | 361 | swvfrpmgtv lllcarpela aplhpgplrr rkplscdvpp aperpavaip twillppssq |
| | | 421 | snasllpvgg nfspkdlfre pgsqsealrc gprvspvtlg vclvvagpla vavglcfspr |
| | | 481 | aepclftpvp gqpcglprgf sraraspapg argapssagg rspstgaars pdgrltlgfv |
| | | 541 | kgprrgfggp lgpprtgsqs rgpgrrsvga arfllrkeld sslpgssasq lsappvqpss |
| | | 601 | wgpavswgsm armnrpapve vsyksmrfli thnptnatls tfiedlkkyg attvvrvcev |
| | | 661 | tydkaplekd gitvvwpgya dpaarlatpc tapgplsfgn rmnprtsgev pavaepgvsp |
| | | 721 | ppflgpflvg nrpgrvdwpf ddgapppgkv vedwlsllka kfcddpgscv avhcvaglgr |
| | | 781 | apvlvalali esgmkyedai qfirqkrrga inskqltyle kyrpkqrlrf kephahktkc |
| | | 841 | cvm |
| SEQ ID NO: 90 | XP_418417.1 | 1 | marmnrpapv evcyknmrfl ithnptnatl stflecngas yrylspagip svvriaicfl |
| | (Gallus gallus) | 61 | gqdiwrtlsi gfvnpiaspt sslkalhkcs lpcifqhdlk kygattvvrv cevtydktpl |
| | (Red Jungle Fowl) | 121 | ekdgitvmdw pfddgappps kivedwinil ktkfcedpgc cvavhcvagl grapvlvala |
| | | 181 | liesgmkyed aiqfirqkrr gainskqlty lekyrpkqrl rfkdphnhkn kccim |
| SEQ ID NO: 91 | XM_519985.1 | 1 | MARMNRPAPV EVSYKHMRFL ITHNPTNATL STFIEDLKKY GATTVVRVCE VTYDKTPLEK |
| | (Pan troglodytes) | 61 | DGITVVDWPF DDGAPPPGKV VEDWLSLVKA KFCEAPGSCV AVHCVAGLGR RSQRGCLGGS |
| | (Chimpanzee) | 121 | QDLDPVLRLG CEAVASMGEL QAGGSLGPLG PRALHLQQVP CQGDRGAQAP MTPALLFAPR |
| | | 181 | APVLVALALI ESGMKYEDAI QFIRQKRRGA INSKQLTYLE KYRPKQRLRF KEPHTHKTRC |
| | | 241 | CVM |

1. A method of culturing a rabbit-mouse hybridoma comprising culturing the hybridoma in a medium comprising IL-6 or a functional equivalent thereof.

2. The method of claim 1, wherein the medium further comprises at least about 1% to about 20% V/V fetal calf serum.

3. A method of producing at least one stable rabbit-mouse hybridoma comprising:
combining rabbit immunocytes with mouse myeloma cells;
subjecting the rabbit immunocytes and mouse myeloma cells to conditions that promote the formation of at least one hybridoma;
and culturing the at least one hybridoma in a medium comprising IL-6 or a functional equivalent thereof, thereby producing at least one stable rabbit-mouse hybridoma.

4. The method of claim 3, wherein the medium is growth medium comprising at least about 1% to about 20% V/V fetal calf serum.

5. The method of claim 3, wherein the medium is selection medium comprising at least about 1% to about 20% V/V fetal-calf serum and HAT.

6. The method of claim 3, further comprising transferring the at least one stable rabbit-mouse hybridoma to a growth medium lacking IL-6 or a functional equivalent thereof.

7. The method of claim 3, wherein the medium comprises supernatant from a cell culture that secretes IL-6 or a functional equivalent thereof.

8. The method of claim 7, wherein the cell culture that secretes IL-6 or a functional equivalent thereof is AMJ2-C11 (AT(C CRL-2456).

9. A stable rabbit-mouse hybridoma produced by the method of claim 3.

10. A rabbit monoclonal antibody secreted by the stable rabbit-mouse hybridoma of claim 9.

11. The rabbit monoclonal antibody of claim 10, wherein the rabbit monoclonal antibody specifically binds to PRL-3.

12. A stable rabbit-mouse hybridoma, wherein the stable rabbit-mouse hybridoma is clone #1.

13. A rabbit monoclonal antibody secreted by the stable rabbit-mouse hybridoma of claim 12.

14. A rabbit monoclonal antibody that specifically binds to PRL-3.

15. The method of claim 1, wherein the hybridoma is produced by fusing rabbit immunocytes with FO (ATCC CRL 1646) myeloma cells.

16. An isolated polypeptide comprising an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, and SEQ ID NO: 13 and variants thereof.


SEQUENCE LISTING (100 sequences)

SEQ ID NO: 1 - protein, 12 aa, Unknown Organism
  Description of Unknown Organism  Human or Mouse epitope
PLEKDGITVVDW

SEQ ID NO: 2 - protein, 12 aa, Unknown Organism
  Description of Unknown Organism Human or Mouse epitope
INSKQLTYLEKY

SEQ ID NO: 3 - protein, 13 aa, Unknown Organism
  Description of Unknown Organism Human, Chimpanzee, or Canine peptide
PTNATLSTFIEDL

SEQ ID NO: 4 - protein, 8 aa, Unknown Organism
  Description of Unknown Organism Human, Chimpanzee, or Canine peptide
NATLSTFI

SEQ ID NO: 5 - protein, 16 aa, Unknown Organism
  Description of Unknown Organism Human, Mouse, or Canine peptide
DGITVVDWPFDDGAPP

SEQ ID NO: 6 - protein, 7 aa, Unknown Organism
  Description of Unknown Organism Human, Mouse, or Canine peptide
DWPFDDG

SEQ ID NO: 7 - protein, 14 aa, Unknown Organism
  Description of Unknown Organism Human, Mouse, or Canine peptide
PPPGKVVEDWLSLV

SEQ ID NO: 8 - protein, 6 aa, Unknown Organism
  Description of Unknown Organism Human, Mouse, or Canine peptide
KVVEDW

SEQ ID NO: 9 - protein, 18 aa, Unknown Organism
  Description of Unknown Organism Human, Mouse, or Canine peptide
GAINSKQLTYLEKYRPKQ

SEQ ID NO: 10 - protein, 6 aa, Unknown Organism
  Description of Unknown Organism Human, Mouse, or Canine peptide
TYLEKY

SEQ ID NO: 11 - protein, 13 aa, Unknown Organism
  Description of Unknown Organism Human, Mouse, or Canine peptide
CEVTYDKTPLEKD

SEQ ID NO: 12 - protein, 7 aa, Unknown Organism
  Description of Unknown Organism Human, Mouse, or Canine peptide
TYDKTPL

SEQ ID NO: 13 - protein, 5 aa, Unknown Organism
  Description of Unknown Organism Human, Mouse, or Canine peptide
PLEKD

SEQ ID NO: 14 - protein, 12 aa, Artificial Sequence
  Description of Artificial Sequence Synthetic peptide
MARMNRPAPVEV

SEQ ID NO: 15 - protein, 12 aa, Artificial Sequence
  Description of Artificial Sequence Synthetic peptide
APVEVSYKHMRF

SEQ ID NO: 16 - protein, 12 aa, Artificial Sequence
  Description of Artificial Sequence Synthetic peptide
KHMRFLITHNPT

SEQ ID NO: 17 - protein, 12 aa, Artificial Sequence
  Description of Artificial Sequence Synthetic peptide
THNPTNATLSTF

SEQ ID NO: 18 - protein, 12 aa, Artificial Sequence
  Description of Artificial Sequence Synthetic peptide
TLSTFIEDLKKY

SEQ ID NO: 19 - protein, 12 aa, Artificial Sequence
  Description of Artificial Sequence Synthetic peptide
DLKKYGATTVVR

SEQ ID NO: 20 - protein, 12 aa, Artificial Sequence
  Description of Artificial Sequence Synthetic peptide
GKVVEDWLSLVK

SEQ ID NO: 21 - protein, 12 aa, Artificial Sequence
  Description of Artificial Sequence Synthetic peptide
AGLGRAPVLVAL

SEQ ID NO: 22 - protein, 12 aa, Artificial Sequence
  Description of Artificial Sequence Synthetic peptide
EDAIQFIRQKRR

SEQ ID NO: 23 - protein, 12 aa, Artificial Sequence
  Description of Artificial Sequence Synthetic peptide
RQKRRGAINSKQ

SEQ ID NO: 24 - protein, 12 aa, Artificial Sequence
  Description of Artificial Sequence Synthetic peptide
KQRLRFKDPHTH

SEQ ID NO: 25 - protein, 12 aa, Artificial Sequence
  Description of Artificial Sequence Synthetic peptide
DPHTHKTRCCVM

SEQ ID NO: 26 - protein, 12 aa, Homo sapiens
MARMNRPAPVEV

SEQ ID NO: 27 - protein, 12 aa, Homo sapiens
APVEVSYKHMRF

SEQ ID NO: 28 - protein, 12 aa, Homo sapiens
KHMRFLITHNPT

SEQ ID NO: 29 - protein, 12 aa, Homo sapiens
THNPTNATLSTF

SEQ ID NO: 30 - protein, 12 aa, Homo sapiens
TLSTFIEDLKKY

SEQ ID NO: 31 - protein, 12 aa, Homo sapiens
DLKKYGATTVVR

SEQ ID NO: 32 - protein, 12 aa, Homo sapiens
GKVVEDWLSLVK

SEQ ID NO: 33 - protein, 12 aa, Homo sapiens
AGLGRAPVLVAL

SEQ ID NO: 34 - protein, 12 aa, Homo sapiens
EDAIQFIRQKRR

SEQ ID NO: 35 - protein, 12 aa, Homo sapiens
RQKRRGAINSKQ

SEQ ID NO: 36 - protein, 12 aa, Homo sapiens
KQRLRFKDPHTH

SEQ ID NO: 37 - protein, 12 aa, Homo sapiens
DPHTHKTRCCVM

SEQ ID NO: 38 - protein, 10 aa, Homo sapiens
MARMNRPAPV

SEQ ID NO: 39 - protein, 10 aa, Homo sapiens
MNRPAPVEVS

SEQ ID NO: 40 - protein, 10 aa, Homo sapiens
PAPVEVSYKH

SEQ ID NO: 41 - protein, 10 aa, Homo sapiens
VEVSYKHMRF

SEQ ID NO: 42 - protein, 10 aa, Homo sapiens
SYKHMRFLIT

SEQ ID NO: 43 - protein, 10 aa, Homo sapiens
HMRFLITHNP

SEQ ID NO: 44 - protein, 10 aa, Homo sapiens
FLITHNPTNA

SEQ ID NO: 45 - protein, 10 aa, Homo sapiens
THNPTNATLS

SEQ ID NO: 46 - protein, 10 aa, Homo sapiens
PTNATLSTFI

SEQ ID NO: 47 - protein, 10 aa, Homo sapiens
ATLSTFIEDL

SEQ ID NO: 48 - protein, 10 aa, Homo sapiens
STFIEDLKKY

SEQ ID NO: 49 - protein, 10 aa, Homo sapiens
IEDLKKYGAT

SEQ ID NO: 50 - protein, 10 aa, Homo sapiens
LKKYGATTVV

SEQ ID NO: 51 - protein, 10 aa, Homo sapiens
YGATTVVRVC

SEQ ID NO: 52 - protein, 10 aa, Homo sapiens
TTVVRVCEVT

SEQ ID NO: 53 - protein, 10 aa, Homo sapiens
VRVCEVTYDK

SEQ ID NO: 54 - protein, 10 aa, Homo sapiens
CEVTYDKTPL

SEQ ID NO: 55 - protein, 10 aa, Homo sapiens
TYDKTPLEKD

SEQ ID NO: 56 - protein, 10 aa, Homo sapiens
KTPLEKDGIT

SEQ ID NO: 57 - protein, 10 aa, Homo sapiens
LEKDGITVVD

SEQ ID NO: 58 - protein, 10 aa, Homo sapiens
DGITVVDWPF

SEQ ID NO: 59 - protein, 10 aa, Homo sapiens
TVVDWPFDDG

SEQ ID NO: 60 - protein, 10 aa, Homo sapiens
DWPFDDGAPP

SEQ ID NO: 61 - protein, 10 aa, Homo sapiens
DDGAPPPGKV

SEQ ID NO: 62 - protein, 10 aa, Homo sapiens
PPPGKVVEDW

SEQ ID NO: 63 - protein, 10 aa, Homo sapiens
KVVEDWLSLV

SEQ ID NO: 64 - protein, 10 aa, Homo sapiens
DWLSLVKAKF

SEQ ID NO: 65 - protein, 10 aa, Homo sapiens
LVKAKFCEAP

SEQ ID NO: 66 - protein, 10 aa, Homo sapiens
KFCEAPGSCV

SEQ ID NO: 67 - protein, 10 aa, Homo sapiens
APGSCVAVHC

SEQ ID NO: 68 - protein, 10 aa, Homo sapiens
CVAVHCVAGL

SEQ ID NO: 69 - protein, 10 aa, Homo sapiens
HCVAGLGRAP

SEQ ID NO: 70 - protein, 10 aa, Homo sapiens
GLGRAPVLVA

SEQ ID NO: 71 - protein, 10 aa, Homo sapiens
APVLVALALI

SEQ ID NO: 72 - protein, 10 aa, Homo sapiens
VALALIESGM

SEQ ID NO: 73 - protein, 10 aa, Homo sapiens
LIESGMKYED

SEQ ID NO: 74 - protein, 10 aa, Homo sapiens
GMKYEDAIQF

SEQ ID NO: 75 - protein, 10 aa, Homo sapiens
EDAIQFIRQK

SEQ ID NO: 76 - protein, 10 aa, Homo sapiens
QFIRQKRRGA

SEQ ID NO: 77 - protein, 10 aa, Homo sapiens
QKRRGAINSK

SEQ ID NO: 78 - protein, 10 aa, Homo sapiens
GAINSKQLTY

SEQ ID NO: 79 - protein, 10 aa, Homo sapiens
SKQLTYLEKY

SEQ ID NO: 80 - protein, 10 aa, Homo sapiens
TYLEKYRPKQ

SEQ ID NO: 81 - protein, 10 aa, Homo sapiens
KYRPKQRLRF

SEQ ID NO: 82 - protein, 10 aa, Homo sapiens
KQRLRFKDPH

SEQ ID NO: 83 - protein, 10 aa, Homo sapiens
RFKDPHTHKT

SEQ ID NO: 84 - protein, 10 aa, Homo sapiens
PHTHKTRCCV

SEQ ID NO: 85 - protein, 28 aa, Artificial Sequence
  Description of Artificial Sequence Synthetic peptide
ATTVVRVCEVTYDKTPLEKDGITVVDWP

SEQ ID NO: 86 - protein, 12 aa, Unknown Organism
  Description of Unknown Organism Human or chicken epitope
THNPTNATLSTF

SEQ ID NO: 87 - protein, 148 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRKRRGAINSKQLTYLEKYRPKQRLRFKDPHTHKTRCCVM

SEQ ID NO: 88 - protein, 173 aa, Mus musculus
MARMNRPAPVEVSYRHMRFLITHNPSNATLSTFIEDLKKYGATTVVRVCEVTYDKTPLEKDGITVVDWPFDDGAPPPGKVVEDWLSLLKAKFYNDPGSCVAVHCVAGLGRAPVLVALALIESGMKYEDAIQFIRQKRRGAINSKQLTYLEKYRPKQRLRFKDPHTHKTRCCVM

SEQ ID NO: 89 - protein, 843 aa, Canis familiaris
MPLFCVAVAVEGEEPFSQGACSGPSQAGAVPAPGLTRGAGGQPPQASGSPRLHEREPGARALESPSPRSRTPALPQPRRPAAQRCSAPGLPGPGLRRRPCASTGPGAGPWPLGPLAPAAPALPARLGTEDARAPSGSRGTHRTFACSAAGGRGGAAEPGDLLGAALRATRSAPDPPSWGLTSGPRRGSFWAKRRGAHACRGVLVGPAPARGPYGTCCDPCERYHLVGTWMLISSIPSISSIPSNSSIPGNPSIPSIPGNPSIPSIPGNTSIPSIPSIPGNPSNSSIPGNPSIPSIPGNPASPASPATPVSPAEWDTWRHQGKPSGGNEAQSLAPLVVPGGFSQCRQVQQVSRPPAADPALSWVFRPMGTVLLLCARPELAAPLHPGPLRRRKPLSCDVPPAPERPAVALPTWILLPPSSQSNASLLPVGGNFSPKDLFREPGSQSEALRCGPRVSPVTLGVCLVVAGPLAVAVGLCFSPRAEPCLFTPVPGQPCGLPRGFSRARASPAPGARGAPSSAGGRSPSTGAARSPDGRLTLGFVKGPRRGFGGPLGPPRTGSQSRGPGRRSVGAARFLLRKELDSSLPGSSASQLSAPPVQPSSWGPAVSWGSMARMNRPAPVEVSYKSMRFLITHNPTNATLSTFIEDLKKYGATTVVRVCEVTYDKAPLEKDGITVVWPGYADPAARLATPCTAPGPLSFQNRMNPRTSGEVPAVAEPGVSPPPFLGPFLVGNRPGRVDWPFDDGAPPPGKVVEDWLSLLKAKFCDDPGSCVAVHCVAGLGRAPVLVALALIESGMKYEDAIQFIRQKRRGAINSKQLTYLEKYRPKQRLRFKEPHAHKTKCCVM

SEQ ID NO: 90 - protein, 235 aa, Gallus gallus
MARMNRPAPVEVCYKNMRFLITHNPTNATLSTFLECNGASYRYLSPAGIPSVVRIAICFLGQDIWRTLSIGFVNPIASPTSSLKALHKCSLPCIFQHDLKKYGATTVVRVCEVTYDKTPLEKDGITVMDWPFDDGAPPPSKIVEDWLNLLKTKFCEDPGCCVAVHCVAGLGRAPVLVALALIESGMKYEDAIQFIRQKRRGAINSKQLTYLEKYRPKQRLRFKDPHNHKNKCCIM

SEQ ID NO: 91 - protein, 243 aa, Pan troglodytes
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRRSQRGCLGGSQDLDPVLRLGCEAVASMGELQAGGSLGPLGPRALHLQQVPCQGDRGAQAPMTPALLFAPRAPVLVALALIESGMKYEDAIQFIRQKRRGAINSKQLTYLEKYRPKQRLRFKEPHTHKTRCCVM

SEQ ID NO: 92 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 93 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 94 - protein, 207 aa, Canis familiaris
MNSLSTSAFSLGLLLVMATAFPTPGPLAGDSKDDATSNSLPLTSANKVEELIKYILGKISALRKEMCDKFNKCEDSKEALAENNLHLPKLEGKDGCFQSGFNQETCLTRITTGLVEFQLHLNILQNNYEGDKENVKSVHMSTKILVQMLKSKVKNQDEVTTPDPTTDASLQAILQSQDECVKHTTIHLILRSLEDFLQFSLRAVRIM

SEQ ID NO: 95 - protein, 208 aa, Bos taurus
MNSRFTSAFTPFAVSLGLLLVMTSAFPTPGPLGEDFKNDTTPGRLLLTTPEKTEALIKRMVDKISAMRKEICEKNDECESSKETLAENKLNLPKMEEKDGCFQSGFNQAICLIRTTAGLLEYQIYLDYLQNEYEGNQENVRDLRKNIRTLIQILKQKIADLITTPATNTDLLEKMQSSNEWVKNAKIILILRNLENFLQFSLRAIRMK

SEQ ID NO: 96 - protein, 211 aa, Rattus rattus
MKFLSARDFQPVAFLGLMLLTATAFPTSQVRRGDFTEDTTHNRPVYTTSQVGGLITYVLREILEMRKELCNGNSDCMNSDDALSENNLKLPEIQRNDGCFQTGYNQEICLLKICSGLLEFRFYLEFVKNNLQDNKKDKARVIQSNTETLVHIFKQEIKDSYKIVLPTPTSNALLMEKLESQKEWLRTKTIQLILKALEEFLKVTMRSTRQT

SEQ ID NO: 97 - protein, 208 aa, Ovis aries
MNSLFTSAFSPLAVSLGLLLVMTSAFPTPGPLGEDFKNDTTPSRLLLTTPEKTEALIKHIVDKISAIRKEICEKNDECENSKETLAENKLKLPKMEEKDGCFQSGFNQAICLIKTTAGLLEYQIYLDFLQNEFEGNQETVMELQSSIRTLIQILKEKIAGLITTPATHTDMLEKMQSSNEWVKNAKVIIILRSLENFLQFSLRAIRMK

SEQ ID NO: 98 - protein, 207 aa, Felis catus
MNFLSTSAFSPLAFSLGLLLVVATAFPTPGPLGGDATSNRLPLTPADKMEELIKYILGKISALKKEMCDNYNKCEDSKEALAENNLNLPKLAEKDGCFQSGFNQETCLTRITTGLQEFQIYLKFLQDKYEGDKENAKSVYTSTNVLLQMLKRKGKNQDEVTIPVPTVEVGLQLSCSHRRVAEAHNNHLTLRRLEDFLQLRLRAVRIM

SEQ ID NO: 99 - protein, 241 aa, Gallus gallus
MNFTEGCEATGRRPGSAGSRRRRAPRPGPVALLPLLLPLLLPPAAAVPLPAAADSSGEVGLEEEAGARRALLDCEPLARVLRDRAVQLQDEMCKKFTVCENSMEMLVRNNLNLPKVTEEDGCLLAGFDEEKCLTKLSSGLFAFQTYLEFIQETFDSEKQNVESLCYSTKHLAATIRQMVINPDEVVIPDSAAQKSLLANLKSDKDWIEKITMHLILRDFTSFMEKTVRAVRYLKKTRSFSA

SEQ ID NO: 100 - protein, 127 aa, Meriones unguiculatus
ENNLKLPEIQRDDGCFHTGYNRDVCLLKITSGLLEYQTYLEYVKNNLQDNKKDKARAIQSNTKTLIRIFKQEVKDPGQIVFPDPTSEALLLEKLESQTEWLKSKTTQLILTALEEFLKVTKRFTLQN